(19) United States
(12) Patent Application Publication
(10) Pub. No.: US 2013/0172252 A1
(43) Pub. Date: Jul. 4, 2013

(54) Methods for promoting hair growth

(71) Applicant: Stemnion, Inc., Pittsburgh, PA (US)

(72) Inventors: George L. Sing, New York, NY (US); Vivienne S. Marshall, San Antonio, TX (US); Charlotte Emig, Gibsonia, PA (US); Martin C. Robson, Stuart, FL (US)

(73) Assignee: STEMNION, INC., Pittsburgh, PA (US)

(21) Appl. No.: 13/777,375

(22) Filed: Feb. 26, 2013

Related U.S. Application Data
(62) Division of application No. 12/452,632, filed on Jan. 12, 2010.

(30) Foreign Application Priority Data
Jul. 21, 2008 (US) PCT/US2008/008841

(51) Int. Cl. A61K 38/18; A61K 45/06; A61K 38/57

(52) U.S. Cl. CPC A61K 38/1866; A61K 38/1891; A61K 38/1858; A61K 38/1841; A61K 38/57; A61K 45/06
USPC 514/8.1

(57) ABSTRACT

The invention is directed to methods for promoting hair growth. Such methods utilize novel compositions, including but not limited to extraembryonic cytokine secreting cells (herein referred to as ECS cells), including, but not limited to, amnion-derived multipotent progenitor cells (herein referred to as AMP cells), conditioned media derived therefrom (herein referred to as amnion-derived cellular cytokine solution or ACCS), cell lysates derived therefrom, and cell products derived therefrom, each alone or in combination.

### CROSS-REFERENCE TO RELATED APPLICATION

This application claims priority under 35 USC §119(e) of U.S. Provisional Application No. 60/961,772, filed Jul. 24, 2007, the entirety of which is incorporated herein by reference.

### BACKGROUND OF THE INVENTION

1. Field of the Invention

The field of the invention is directed to methods for promoting hair growth. Such methods utilize novel compositions, including but not limited to extraembryonic cytokine secreting cells (herein referred to as ECS cells), including, but not limited to, amnion-derived multipotent progenitor cells (herein referred to as AMP cells), conditioned media derived therefrom (herein referred to as amnion-derived cellular cytokine solution or ACCS), cell lysates derived therefrom, and cell products derived therefrom, each alone or in combination.

2. Related Art

Philp, D., et al., (The FASEB J express article 10.1096/fj.03-0244fje, published online Dec. 4, 2003) report that Thymosin β4 increases hair growth by activation of hair follicle stem cells.

In humans, each hair follicle undergoes repeated cyclical periods of growth. These cycles include anagen, an active growth stage which can last for ˜2 to 6 years; catagen, a transition phase, which lasts for only ˜1-2 weeks; and telogen, a resting period which lasts ˜3-4 months after which the hair is shed and a new hair is grown as the cycle repeats itself. In the normal human scalp, which contains approximately 100,000 hair follicles, ˜86% of the hair follicles are in anagen, ˜1% are in catagen, and ˜13% are in telogen. Thus, under normal conditions, approximately 100 hairs are shed from the scalp each day.

Androgens (steroid hormones such as estrogen and testosterone) are the most obvious regulators of human hair growth in both sexes. Interestingly, androgens have contrasting effects on hair follicles depending on the hair follicle's location in the body. Androgens stimulate hair growth in many locations (i.e., beard, axilla) while inhibiting scalp hair growth in genetically predisposed individuals. Androgens act on the hair follicles via the dermal papilla, presumably by altering the production of regulatory factors that influence the dermal papilla cells. Cultured dermal papilla cells secrete factors which are mitogenic for other dermal papilla cells, outer root sheath cells, epidermal keratinocytes and endothelial cells. Androgen-sensitive cells from beard or balding scalp reflect their in vivo androgenetic responses by responding to testosterone by either increasing (i.e., beard) or decreasing (i.e., balding) their mitogenic ability.

Excessive hair loss, or alopecia, occurs in many people for a variety of reasons. Alopecia can be classified as being one of two types: non-scarring alopecia and scarring alopecia. Non-scarring alopecia has been attributed to genetics and advanced age (i.e. androgenetic alopecia); administration of drugs such as anti-cancer chemotherapeutic drugs and contraceptives; topical use of chemical treatments, such as hair dyes, permanent wave solutions, etc.; diseases, such as leprosy or syphilis; illness; allergy; and hair follicle infection. Scarring alopecia may be a consequence of burns (accidental or post surgical from cryosurgery or laser surgery) or trauma, which often causes destruction of hair follicles.

Despite the widespread occurrence of alopecia, especially androgenetic alopecia, the need for prevention and therapy still exists. Current remedies include wearing of wigs or toupees; surgery including hair transplant surgery; scalp reduction and scalp flaps; topical drugs such as minoxidil (Rogaine™); oral medications such as finasteride, (Propecia™), SKF-105657, cyproterone acetate, and duasteride (Avodart™); corticosteroids; and various herbal remedies. Therefore, it is an object of the invention described herein to meet this unmet need.

### BRIEF SUMMARY OF THE INVENTION

It is an object of the instant invention to provide novel methods useful in promoting hair growth. Such methods utilize novel compositions, including but not limited to extraembryonic cytokine secreting cells (herein referred to as ECS cells), conditioned media derived therefrom, cell lysates derived therefrom, and cell products derived therefrom. In a specific embodiment, the ECS cells include, but are not limited to, amnion-derived multipotent progenitor cells (herein referred to as AMP cells), conditioned media derived therefrom (herein referred to as amnion-derived cellular cytokine solution or ACCS), cell lysates derived therefrom, and cell products derived therefrom, each alone and/or in combination with each other and/or with other agents including active and/or inactive agents.

Accordingly, a first aspect of the invention is a method for promoting hair growth in a subject in need thereof comprising administering to the subject a therapeutically effective amount of one or more compositions comprising ECS cells, conditioned media derived therefrom, cell lysate derived therefrom or cell products derived therefrom.

A second aspect of the invention is a method for stimulating hair follicle stem cell differentiation in a subject in need thereof comprising administering to the subject a therapeutically effective amount of one or more compositions comprising ECS cells, conditioned media derived therefrom, cell lysate derived therefrom or cell products derived therefrom.

In a specific embodiment of the invention, the ECS cells are AMP cells. In another specific embodiment the AMP cells are pooled AMP cells.

In another specific embodiment of the invention, the conditioned media is ACCS. In still another specific embodiment the ACCS is pooled ACCS.

In a third aspect of the invention, the ECS cells, conditioned media derived therefrom, cell lysate derived therefrom or cell products derived therefrom are administered in combination with each other and/or other agents or therapies. In a specific embodiment, the other agents are active agents. In particular embodiments, the active agents are minoxidil or finasteride.

In a fourth aspect of the invention, ECS cells are undifferentiated, partially differentiated, fully differentiated or a combination thereof. In a particular embodiment, the AMP cells are partially differentiated or fully differentiated.

Other features and advantages of the invention will be apparent from the accompanying description, examples and the claims. The contents of all references, pending patent applications and published patents, cited throughout this application are hereby expressly incorporated by reference. In case of conflict, the present specification, including definitions, will control.

### DEFINITIONS

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of the plasma membrane of a cell or in some cases of a specific cell type.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cytokine secreting cells” or “ECS cells” means a population of cells derived from the extraembryonic tissue which have the characteristic of secreting a unique combination of physiologically relevant cytokines in a physiologically relevant temporal manner into the extracellular space or into surrounding culture media. In one embodiment, the ECS cells secrete at least one cytokine selected from VEGF, Angiogenin, PDGF and TGFβ2 and at least one MMP inhibitor selected from TIMP-1 and TIMP-2. In another embodiment, the ECS cells secrete more than one cytokine selected from VEGF, Angiogenin, PDGF and TGFβ2 and more than one MMP inhibitor selected from TIMP-1 and TIMP-2. In a preferred embodiment, the ECS cells secrete the cytokines VEGF, Angiogenin, PDGF and TGFβ2 and the MMP inhibitors TIMP-1 and TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg mL for TIMP-1 and ˜1.04 μg/mL for TIMP-2. ECS cells also secrete Thymosin β4 protein. ECS cells may be selected from populations of cells and compositions described in this application and in US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179, the contents of which are incorporated herein by reference in their entirety.

As used herein, the term “amnion-derived multipotent progenitor cell” or “AMP cell” means a specific population of ECS cells that are epithelial cells derived from the amnion. In addition to the characteristics described above for ECS cells, AMP cells have the following characteristics. They have not been cultured in the presence of any animal-derived products, making them suitable for human clinical use. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated AMP cells will not react with antibodies to the stem/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells. AMP cells have previously been described as “amnion-derived cells” (see U.S. Provisional Application Nos. 60/666,949, 60/699,257, 60/742,067, U.S. Provisional Application Nos. 60/813,759, U.S. application Ser. No. 11/333,849, U.S. application Ser. No. 11/392,892, and PCTUS06/011392, each of which is incorporated herein in its entirety).

By the term “animal-free” when referring to certain compositions, growth conditions, culture media, etc. described herein, is meant that no animal-derived materials, such as animal-derived serum, other than human materials, such as native or recombinantly produced human proteins, are used in the preparation, growth, culturing, expansion, or formulation of the certain composition or process.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, the term “differentiation” means the process by which cells become progressively more specialized.

As used herein, the term “differentiation efficiency” means the percentage of cells in a population that are differentiating or are able to differentiate.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein. As used herein, conditioned medium also refers to components, such as proteins, that are recovered and/or purified from conditioned medium or from ECS cells, including AMP cells.

As used herein, the term “amnion-derived cellular cytokine solution” or “ACCS” means conditioned medium that has been derived from AMP cells or expanded AMP cells. Amnion-derived cellular cytokine solution has previously been referred to as “amnion-derived cellular cytokine suspension”.

The term “physiological level” as used herein means the level that a substance in a living system is found and that is relevant to the proper functioning of a biochemical and/or biological process.

As used herein, the term “pooled” means a plurality of compositions that have been combined to create a new composition having more constant or consistent characteristics as compared to the non-pooled compositions. For example, pooled ACCS have more constant or consistent characteristics compared to non-pooled ACCS.

The term “therapeutically effective amount” means that amount of a therapeutic agent necessary to achieve a desired physiological effect (i.e. stimulating hair growth).

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases.

As used herein, the term “substrate” means a defined coating on a surface that cells attach to, grown on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “transplantation” as used herein refers to the administration of a composition comprising cells that are either in an undifferentiated, partially differentiated, or fully differentiated form into a human or other animal.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, the term “hair follicle” means a tube-like opening in the epidermis where the hair shaft develops and into which the sebaceous glands open.

As used herein, the term “temporal expression” means expression of a gene or protein which is limited in time, temporary, or transient.

### BRIEF DESCRIPTION OF THE FIGURES

FIG. 1. Hair growth is visible in the animal treated with ACCS.

FIG. 2. No hair growth is seen in the animal treated with saline.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Therapeutic Uses

The most common type of human hair loss is androgenetic alopecia (also known as androgenic alopecia), which is a loss of telogen hairs caused by an excessive androgen effect in genetically susceptible men and women. Androgens trigger the miniaturization or atrophy of the hair follicles which normally produce thick scalp hair and transforms them into vellus-like hair follicles which yield fine, downy hair that is barely perceptible. Androgenetic alopecia is expressed in males as baldness of the vertex of the scalp and is commonly referred to as male pattern baldness. In females, androgenetic alopecia appears as diffuse hair loss or thinning of the front, top and sides areas. As alopecia progresses with age, hairs in these predisposed areas miniaturize and appear to change from terminal hairs to resemble vellus hairs. In addition, as androgenetic alopecia continues, the number of hairs in the active growth anagen phase decreases while there is an increase the number of hairs in the telogen phase.

It is currently believed that the conversion of testosterone into dihydrotestosterone, a compound which inhibits hair growth, by the enzyme 5-α-reductase, triggers pattern baldness in men, but the mechanism of interaction between the hormone and hair follicles remains unknown. Female pattern baldness is thought to result from a decrease in estrogen, a hormone that normally counteracts the balding effect of testosterone, although there is so far no consensus on whether pattern baldness in women is truly androgen-dependent.

Another common type of hair loss is alopecia greata, an autoimmune disease which afflicts an estimated four million people. Alopecia greata usually presents as varying amounts of patchy hair loss, most commonly on the scalp (though it can affect any hair-bearing surface), but may also manifest as larger patches with little or no hair. Related forms of the disease include: (1) alopecia totalis, characterized by complete loss of all scalp hair; and (2) alopecia universalis, characterized by loss of all body hair, including eyelashes, eyebrows, underarm hair, and pubic hair. The latter form can cause serious respiratory problems because the nostrils and sinuses are no longer protected from airborne foreign particles.

A less common form of hair loss is telogen effluvium, which manifests as excessive shedding of hair because hair follicles prematurely enter telogen. It may be caused by a multitude of stress-related causes, including high fevers, childbirth, severe infections, severe chronic illness, severe psychological stress, major surgery, an over- or under-active thyroid gland, crash diets with inadequate protein, and a variety of medications, including, e.g., retinoids, beta blockers, calcium channel blockers, antidepressants, and non-steroidal anti-inflammatory agents, including ibuprofen and acetominophen. Generally little treatment is possible beyond identifying and either treating or discontinuing the causing factor, whichever is appropriate. In most cases, the lost hair will be replaced within a year or so.

Anagen effluvium, the most common type of chemotherapy-induced alopecia, results from the abrupt cessation of mitotic activity in hair matrix cells of anagen hair follicles. This induces the follicles to produce either no hair, or produce only narrow defective hair sheaths which are predisposed to fracture and loss. This type of alopecia can be seen to some degree in many anti-neoplastic therapies. However, there are certain agents, such as bleomycin, cisplatin, doxorubicin, vinblastine and vincristine, which induce alopecia more frequently and severely. Anagen effluvium manifests within 1 to 2 weeks after the beginning of chemotherapy but is most noticeable 1 to 2 months later. Initially, there may not be total hair loss, since approximately 10% of follicles will not be in anagen phase at the start of chemotherapy. Total hair loss eventually occurs with prolonged therapy, which can also induce hair loss in other areas of the body. Hair regrowth can usually be expected after the end of chemotherapy, although hair color and texture may change.

Compositions for Practicing Methods of the Invention

Obtaining ECS Cells

Various general methods for isolating cells from the extraembryonic tissue, which may then be used to produce the ECS cells useful in practicing the instant invention, are described in the art (see, for example, US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179).

Identifying ECS Cells

Once extraembryonic tissue is isolated, it is necessary to identify which cells in the tissue have the characteristics associated with ECS cells (see definition above). For example, cells are assayed for their ability to secrete a unique combination of cytokines into the extracellular space or into surrounding culture media. Suitable cells are those in which the cytokine or cytokines occurs in the physiological range of ˜5.0-16 ng/mL for VEGF, ˜3.5-4.5 ng/mL for Angiogenin, ˜100-165 pg/mL for PDGF, ˜2.5-2.7 ng/mL for TGFβ2, ˜0.68 μg/mL for TIMP-1 and ˜1.04 μg/mL for TIMP-2. Such cells also secrete Thymosin β4. Philp, D., et al., (The FASEB J express article 10.1096/fj.03-0244fje, published online Dec. 4, 2003) report that Thymosin (34 increases hair growth by activation of hair follicle stem cells.

Obtaining AMP Cells

In a particular embodiment, useful AMP cell compositions are prepared using the steps of a) recovery of the amnion from the placenta, b) dissociation of the cells from the amniotic membrane, c) culturing of the cells in a basal medium with the addition of a naturally derived or recombinantly produced human protein; d) selecting AMP cells from the cell culture, and optionally e) further proliferation of the cells, optionally using additional additives and/or growth factors. Details are contained in US Publication No. 2006-0222634-A1, which is incorporated herein by reference.

AMP May Cells are Cultured as Follows

The AMP cells are cultured in a basal medium. Such medium includes, but is not limited to, Epilife (Cascade Biologicals), Opti-pro, VP-SFM, IMDM, Advanced DMEM, K/O DMEM, 293 SFM II (all made by Gibco; Invitrogen), HPGM, Pro 2935-CDM, Pro 293A-CDM, UltraMDCK, UltraCulture (all made by Cambrex), Stemline I and Stemline II (both made by Sigma-Aldrich), DMEM, DMEM/F-12, Ham's F12, M199, and other comparable basal media. Such media should either contain human protein or be supplemented with human protein. As used herein a “human protein” is one that is produced naturally or one that is produced using recombinant technology. “Human protein” also is meant to include a human fluid or derivative or preparation thereof, such as human serum or amniotic fluid, which contains human protein. Details on this procedure are contained in US Publication No. 2006-0222634-A1, which is incorporated herein by reference.

In a most preferred embodiment, the cells are cultured using a system that is free of animal products to avoid xeno-contamination. In this embodiment, the culture medium is Stemline I or II, Opti-pro, IMDM, or DMEM, with human albumin added up to concentrations of 10%. The invention further contemplates the use of any of the above basal media wherein animal-derived proteins are replaced with recombinant human proteins and animal-derived serum, such as BSA, is replaced with human albumin. In preferred embodiments, the media is serum-free in addition to being animal-free. Details on this procedure are contained in US Publication No. 2006-0222634-A1, which is incorporated herein by reference.

In alternative embodiments, where the use of non-human serum is not precluded, such as for in vitro uses, the culture medium may be supplemented with serum derived from mammals other than humans, in ranges of up to 40%.

Additional Proliferation

Optionally, other proliferation factors are used. In one embodiment, epidermal growth factor (EGF), at a concentration of between 0-1 μg/mL is used. In a preferred embodiment, the EGF concentration is around 10 ng/mL. Alternative growth factors which may be used include, but are not limited to, TGFα or TGFβ (5 ng/mL; range 0.1-100 ng/mL), activin A, cholera toxin (preferably at a level of about 0.1 μg/mL; range 0-10 μg/mL), transferrin (5 μg/mL; range 0.1-100 μg/mL), fibroblast growth factors (bFGF 40 ng/mL (range 0-200 ng/mL), aFGF, FGF-4, FGF-8; (all in range 0-200 ng/mL), bone morphogenic proteins (i.e. BMP-4) or other growth factors known to enhance cell proliferation.

Generation of Conditioned Medium

ECS Conditioned Medium

ECS conditioned medium is obtained as described below for ACCS, except that ECS cells are used.

Generation of ACCS

The AMP cells can be used to generate ACCS. In one embodiment, the AMP cells are isolated as described herein and 1×10⁶ cells/mL are seeded into T75 flasks containing between 5-30 mL culture medium, preferably between 10-25 mL culture medium, and most preferably about 10 mL culture medium. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 4 days post-confluence. In another embodiment the medium is changed and ACCS is collected 5 days post-confluence. In a preferred embodiment the medium is changed and ACCS is collected 3 days post-confluence. In another preferred embodiment the medium is changed and ACCS is collected 3, 4, 5, 6 or more days post-confluence. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release following collection. Skilled artisans are familiar with cryopreservation, lyophilization, and sustained-release methodologies.

The compositions useful in practicing the invention can be prepared in a variety of ways. For example, a composition useful in practicing the invention may be a liquid comprising an agent of the invention, i.e. ECS cells, including AMP cells and/or ACCS, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like, suitable for topical administration.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and muco-adhesive.

Pharmaceutical Compositions

The present invention provides pharmaceutical compositions of ECS cells, including AMP cells and/or ACCS and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Treatment Kits

The invention also provides for an article of manufacture comprising packaging material and a pharmaceutical composition of the invention contained within the packaging material, wherein the pharmaceutical composition comprises compositions of ECS cells, including AMP cells and/or ACCS. The packaging material comprises a label or package insert which indicates that the ECS cells, including AMP cells and/or ACCS can be used for promoting hair growth.

Formulation, Dosage and Administration

Compositions comprising ECS cells, including AMP cells and/or ACCS may be administered to a subject to provide various cellular or tissue functions, for example, to promote hair growth. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may be packaged with written instructions for their use in promoting hair growth. The compositions may also be administered to the recipient in one or more physiologically acceptable carriers. Carriers for the cells may include but are not limited to solutions of phosphate buffered saline (PBS), normal saline or lactated Ringer's solution containing a mixture of salts in physiologic concentrations, or cell culture medium.

One of skill in the art may readily determine the appropriate concentration, or dose, of the ECS cell conditioned media, including ACCS, for a particular purpose. The skilled artisan will recognize that a preferred dose is one which produces a therapeutic effect, such as promoting hair growth, in a subject in need thereof. For example, one preferred dose of ACCS is in the range of about 0.1-to-1000 μL per square centimeter of applied area. Other preferred dose ranges are 1.0-100 μL per square centimeter of applied area and about 0.01-to-50.0 μL per square centimeter of applied area. Of course, proper doses of ECS cell conditioned media, including ACCS, will require empirical determination at time of use based on several variables including but not limited to the severity of disease, disorder or condition being treated; patient age, weight, sex, health; other medications and treatments being administered to the patient; and the like. One of skill in the art will also recognize that number of doses (dosing regimen) to be administered needs also to be empirically determined based on, for example, severity of disease, disorder or condition being treated. In a one embodiment, one dose is sufficient. Other embodiments contemplate, 2, 3, 4, or more doses. Furthermore, conditioned media derived from ECS cells, including ACCS derived from AMP cells, is typically administered at full strength because the cytokines and factors contained therein are present at physiologic levels (see Steed, D. L., et al, Eplasty 2008, Vol. 8, e19, published online Apr. 7, 2008, for a discussion of such physiologic levels of cytokines and factors in ACCS). Again, the volume of conditioned media, including ACCS, will depend upon the extent of injury or disease being treated, etc., and can only be determined by the attending physician at time of use.

One of skill in the art may readily determine the appropriate concentration, or dose, of the ECS cells, including AMP cells, for a particular purpose, as well. The skilled artisan will recognize that a preferred dose is one which produces a therapeutic effect, such as promoting hair growth, in a subject in need thereof. For example, ECS cells, including AMP cells, are prepared at a concentration of between about 1×10⁷-1×10⁸ cells/mL, preferably at about 2.5×10⁷-7.5×10⁷ cells/mL, and most preferably at about 5×10⁷ cells/mL. The volume of cell mixture administered will depend upon several variables and can only be determined by the attending physician at time of use. Such proper doses of the ECS cells, including AMP cells, will require empirical determination based on such variables as the severity and type of disease, injury, disorder or condition being treated; patient age, weight, sex, health; other medications and treatments being administered to the patient; and the like.

The present invention provides for methods of promoting hair growth by administering to a subject ECS cells, including AMP cells and/or ACCS in a therapeutically effective amount. By “therapeutically effective amount” is meant the dose of ECS cells, including AMP cells and/or ACCS that is sufficient to elicit a therapeutic effect. Thus, the concentration of ECS cells, including AMP cells and/or ACCS in an administered dose unit in accordance with the present invention is effective in promoting hair growth.

In further embodiments of the methods of the present invention, at least one additional agent may be combined with the ECS cells, including AMP cells and/or ACCS. Such agents include, for example, minoxidil, finasteride, etc. In addition to these agents, it may be desirable to co-administer other agents, including other active agents and/or inactive agents, with the ECS cells, including AMP cells and/or ACCS. Active agents include but are not limited to growth factors, cytokines, chemokines, other cell types, and the like. Inactive agents include carriers, diluents, stabilizers, gelling agents, delivery vehicles, ECMs (natural and synthetic), scaffolds, and the like. When the ECS cells, including AMP cells and/or ACCS are administered conjointly with other pharmaceutically active agents even less of the ECS cells, including AMP cells and/or ACCS may be needed to be therapeutically effective at promoting hair growth.

ECS cells, including AMP cells and/or ACCS can be administered topically to a target site of a subject, or may be administered by other means. Specific, non-limiting examples of administering AMP cells and/or ACCS to subjects may also include administration by subcutaneous injection, intramuscular injection or intradermal injection.

The timing of administration of ECS cells, including AMP cells and/or ACCS will depend upon the severity of the hair loss condition being treated. In a preferred embodiment, the ECS cells, including AMP cells and/or ACCS, are administered as soon as possible after diagnosis. In other preferred embodiments, the ECS cells, including AMP cells and/or ACCS are administered more than one time following diagnosis.

Also contemplated by the methods of the invention are compositions comprising partially or fully differentiated ECS cells, including AMP cells, or combinations thereof. Such partially or fully differentiated cell compositions are obtained by treating ECS cells, including AMP cells, with appropriate reagents and under appropriate conditions wherein the cells undergo partial or complete differentiation. Skilled artisans are familiar with conditions capable of effecting such partial or complete differentiation. The cells may be treated under differentiating conditions prior to use (i.e. transplantation, administration, etc.), simultaneously with use or post-use. In certain embodiments, the cells are treated under differentiation conditions before and during use, during and after use, before and after use, or before, during and after use. In other embodiments, the undifferentiated, partially differentiated or fully differentiated cells may be admixed prior to administration.

Skilled artisans will recognize that any and all of the standard methods and modalities for promoting hair growth currently in clinical practice and clinical development are suitable for practicing the methods of the invention. Routes of administration, formulation, co-administration with other agents (if appropriate) and the like are discussed in detail elsewhere herein.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Recovery of AMP Cells

AMP cells were dissociated from starting amniotic membrane using the dissociation agents PXXIII, and trypsin. The average weight range of an amnion was 18-27 g. The number of cells recovered per g of amnion was about 10-15×10⁶ for dissociation with PXXIII and 5-8×10⁶ for dissociation with trypsin.

Method of obtaining selected AMP cells: Cells were plated immediately upon isolation from the amnion. After ˜2 days in culture non-adherent cells were removed and the adherent cells were kept. This attachment to plastic tissue culture vessel is the selection method used to obtain the desired population of cells. Adherent and non-adherent AMP cells appear to have a similar cell surface marker expression profile but the adherent cells have greater viability and are the desired population of cells. Adherent AMP cells were cultured until they reached 120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are typically not selected for further analysis and use. Once the AMP cells reach 120,000-150,000 cells/cm², they were collected and cryopreserved. This collection time point is called p0.

### Example 2

### Generation of ACCS

The AMP cells are used to generate ACCS. The AMP cells were isolated as described herein and 1×10⁶/mL cells were seeded into T75 flasks containing 10 mL culture medium. The cells were cultured until confluent, the medium was changed and ACCS was collected 3 days post-confluence. Skilled artisans will recognize that other embodiments for collecting ACCS from confluent cultures, such as using other tissue culture vessels, including but not limited to cell factories, flasks, hollow fibers, or suspension culture apparatus, are also contemplated by the methods of the invention. It is also contemplated by the instant invention that the ACCS be cryopreserved following collection. It is also contemplated that the ACCS be lyophilized following collection. It is also contemplated that the ACCS be formulated for sustained-release following collection.

### Example 3

### Detection of Growth Factors and Cytokines Important in Promoting Hair Growth

To determine which growth factors and/or cytokines important in promoting hair growth may be secreted by the AMP cells of the present invention, ACCS was isolated from cell cultures as described above.

The ACCS was analyzed for secreted factor content via antibody arrays, ELISA, multiplex and mass spectroscopy assays.

Results

The following relevant factors were detected via antibody arrays, ELISA or multiplex assay in ACCS: bFGF (FGF2), PDGF, KGF (low), IGF-1 (low). Thymosin β4 was detected by mass spectroscopy.

### Example 4

### Growth of Hair on Guinea Pigs Following Exposure to AMP Cells and/or ACCS

A modified partial-thickness scald burn model (species—Hartley guinea pig) was used in this experiment because uniform partial-thickness burns cannot be reproducibly created in mice or rats because of their hair cycle (estrus cycle). Guinea pigs do not have hair cycles. Epithelialization, hair growth, and histology can be evaluated with this model.

Methodology

Briefly, under anesthesia, the animals' backs were shaved and depilated and a uniform scald burn over 10% of the body surface was performed at 75° C. for 10 seconds. After cooling to room temperature, the burn wounds were lightly abraded to remove the burned epidermis.

Experimental Groups

The experimental groups were as follows: Group I—untreated as controls; Group II—treated with 0.007 mL/cm² of unconditioned media (UCM) on day 0 (day of debridement) and day 7; Group III—treated with 0.007 mL/cm² ACCS on day 0 and day 7; Group IV-treated with 0.007 mL/cm² UCM and AMP cells (1×10⁶) on day 0 and day 7; Group V—treated with 0.007 mL/cm² ACCS and AMP cells (1×10⁶) on day 0 and day 7. The doses were given once a week (every 7 days) for a total of 14 days.

Analyses

The animals were premedicated, anesthetized and wound tracings of the epithelialized areas performed every five days. Digital planimetry was performed on the tracings. Evaluation of hair growth was made. Burn wound biopsies were obtained on a weekly basis until the time of healing. Histological analyses of the healing skin were made. Gross observations were made and photographically documented for the quality of healing and hair distribution.

Healing Results

Large areas of the burns converted to full-thickness injury and did not epithelialize, especially in the untreated control group where only 40% epithelialization occurred by 15 days. The three groups treated by either ACCS or AMP cells epithelialized significantly better than the controls and Group V, treated with both ACCS and AMP cells, epithelialized the best, reaching 80% healing by day 15.

Hair Growth Results

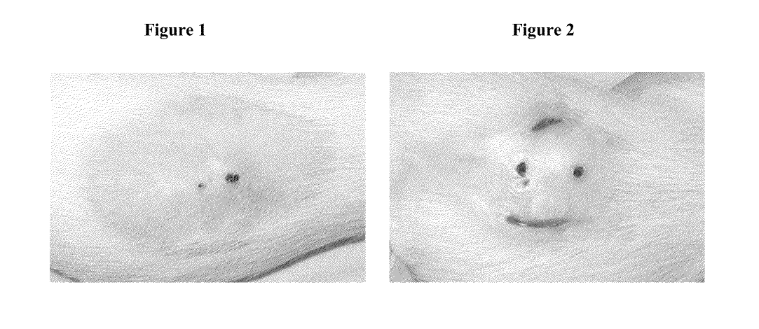

Significantly, hair growth occurred in the animals treated with either ACCS or AMP (FIG. 1) cells but not in the two control groups (FIG. 2).

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

1.-12. (canceled)

13. A method for promoting hair growth in a subject having a condition which would benefit therefrom, the method comprising the step of topically administering to the subject a composition comprising Amnion-derived Cellular Cytokine Solution (ACCS), wherein the ACCS comprises about 5.0-16 ng/mL VEGF, about 3.5-4.5 ng/mL Angiogenin, about 100-165 pg/mL PDGF, about 2.5-2.7 ng/mL TGFβ2, about 0.68 μg/mL TIMP-1 and about 1.04 μg/mL TIMP-2.

14. The method of claim 13 wherein the ACCS is administered in combination with other agents or therapies.

15. The method of claim 14 wherein the other agents are selected from the group consisting of minoxidil and finasteride.

16. A method for stimulating hair follicle stem cell differentiation in a subject having a condition which would benefit therefrom, the method comprising the step of topically administering to the subject a composition comprising ACCS, wherein the ACCS comprises about 5.0-16 ng/mL VEGF, about 3.5-4.5 ng/mL Angiogenin, about 100-165 pg/mL PDGF, about 2.5-2.7 ng/mL TGFβ2, about 0.68 μg/mL TIMP-1 and about 1.04 μg/mL TIMP-2.

17. The method of claim 16 wherein the ACCS is administered in combination with other agents or therapies.

18. The method of claim 17 wherein the other agents are selected from the group consisting of minoxidil and finasteride.

19. A method for stimulating DNA synthesis in a hair follicle in a subject having a condition which would benefit therefrom, the method comprising the step of topically administering to the subject a composition comprising ACCS, wherein the ACCS comprises about 5.0-16 ng/mL VEGF, about 3.5-4.5 ng/mL Angiogenin, about 100-165 pg/mL PDGF, about 2.5-2.7 ng/mL TGFβ2, about 0.68 μg/mL TIMP-1 and about 1.04 μg/mL TIMP-2.

20. The method of claim 19 wherein the ACCS is administered in combination with other agents or therapies.

21. The method of claim 20 wherein the other agents are selected from the group consisting of minoxidil and finasteride.
